(19) United States
(12) Patent Application Publication
(10) Pub. No.: US 2022/0265644 A1
(43) Pub. Date: Aug. 25, 2022

(54) FIXED DOSE COMBINATION DRUG FOR THE TREATMENT OF MALARIA

(71) Applicant: SUN PHARMACEUTICAL INDUSTRIES LTD., Mumbai (IN)

(72) Inventors: Altaf LAL, Gurugram (IN); Amit NASA, Gurugram (IN)

(73) Assignee: SUN PHARMACEUTICAL INDUSTRIES LTD., Mumbai (IN)

(21) Appl. No.: 17/741,952

(22) Filed: May 11, 2022

Related U.S. Application Data
(63) Continuation of application No. PCT/IB2021/057494, filed on Aug. 13, 2021.

(30) Foreign Application Priority Data
Aug. 14, 2020 (IN) 202021035162

(51) Int. Cl. A61K 31/496; A61K 31/357; A61K 31/4709; A61K 31/4706; A61P 33/06

(52) U.S. Cl. CPC A61K 31/496; A61K 31/357; A61K 31/4709; A61K 31/4706; A61P 33/06

(57) ABSTRACT

A composition for treating malaria comprising Arterolane and Piperaquine is disclosed. The Arterolane and Piperaquine are present in an effective amount according a body-weight dosing regimen of a patient.

### CROSS-REFERENCE TO RELATED APPLICATIONS

This application is a bypass continuation of International Application No. PCT/IB2021/057494, filed on Aug. 13, 2021, the disclosures of which are incorporated herein by reference by reference in their entireties.

### FIELD

The present disclosure teaches techniques including a body weight-based dosing regimen for the administration of Arterolane and Piperaquine to a patient for the treatment of malaria. The present disclosure also provides a method for treating malaria in a patient comprising administering to the patient a therapeutically effective amount of Arterolane and Piperaquine in accordance with a body weight-based dosing regimen, wherein the dose of Arterolane ranges from about 3 mg/kg/day to about 6.5 mg/kg/day and that of Piperaquine from about 15 mg/kg to about 32 mg/kg/day. Furthermore, the present disclosure provides a triple combination of Arterolane, Piperaquine and Mefloquine, wherein Arterolane and Piperaquine are dosed in accordance with a body weight-based dosing regimen.

### BACKGROUND

Malaria is an acute and often chronic infectious disease resulting from the presence of protozoan parasites within red blood cells. It is caused by single-celled parasites of the genus Plasmodium, malaria is transmitted from person to person by the bite of female mosquitos. Five species of Plasmodium protozoan parasites are generally responsible for malaria, including Plasmodium vivax, Plasmodium falciparum, Plasmodium malariae, Plasmodium ovale, and Plasmodium knowlesi. Of the five species, Plasmodium falciparum is the most dangerous, accounting for half of all clinical cases of malaria and 90% of deaths from the disease.

The World Health Organization (WHO) recommends Artemisinin-based combination therapies (ACTs) for the treatment of uncomplicated malaria caused by the P. falciparum parasite. The rationale behind the use of combination therapies is that the Artemisinin derivative will rapidly reduce the parasite density because of its high potency antimalarial effect; while the partner drug with a longer half-life will clear the remaining parasites. To cure a patient of malaria, both components of an ACT need to function effectively, so resistance to either drug is critical. The three main ACTs recommended for the treatment of uncomplicated malaria caused by the P. falciparum parasite are (i) Artemether and Lumefantrine, (ii) Artesunate and Amodiaquine, and (iii) Dihydroartemisinin-Piperaquine.

Despite the fact that ACTs are still working in most malaria endemic areas and most patients are cured, the small numbers of patients failing, and the resulting treatment failure now severely threatens treatment of falciparum malaria. In some cases, it has been found that the treatment failure results from drug resistance or inadequate exposure to the drug due to sub-optimal dosing or poor adherence. It has been found that sub-optimal dosing leads to inadequate exposure to the drug, which contributes to emergence of drug resistance to Artemisinins and their partners. Fixed dose combinations (FDCs) encourage adherence and are preferred to lose (individual) tablets.

An FDC of rapidly and short acting Arterolane maleate (150 mg) and slow and long acting Piperaquine phosphate (750 mg) (Synriam®) eliminates the residual parasites. It is commercially available in India and 12 other African countries as an oral tablet composition. It also provides a simplified once-a-day dosing three day therapy for the treatment of acute uncomplicated P. falciparum malaria infection in individuals from 12 to 65 years. This convenient short course treatment may also encourage compliance. Synriam® is also available as an oral dispersible tablet (Synriam® DT) composition comprising Arterolane (37.5 mg) and Piperaquine phosphate (187.5 mg) for the treatment of children aged 6 months to 12 years. In case of Synriam® DT, the dosage is based on the age of the child, for example one tablet containing 37.5 mg of Arterolane and 187.5 mg of Piperaquine phosphate is administered to individuals of age ranges from 6 months to up to 2 years. Individuals whose age ranges from 2 to up to 6 years receive two tablets, while children 6 to 12 years of age receive three tablets once-a-day dosing three day therapy.

The dosage or dosing regimen available to date may result in under-dosing or over-dosing of some patients. Such dosage or dosing regimens may affect exposure to a drug and thus treatment efficacy and emergence of drug resistance. Furthermore, a resistant parasite that evades being killed by sub-optimal antimalarial treatment can propagate, and transmit, facilitating the selection and spread of resistance. Assuring that all patients receive an optimal dose is an important step in slowing the emergence and spread of resistance to these valuable drugs.

Thus, there is a long felt need to provide an alternative therapy in malaria control with a potential role in treatment of resistant malaria and prevention of further development of parasite resistance. The inventors of the present application discovered that the similar exposure across all patient groups is important; hence an alternative fixed dose combination composition providing a new dosing regimen is required.

Thus, the present disclosure provides a great hope for a new dosing regimen to meet the challenges of a serious global public health concern due to P. falciparum malaria infection including resistant malaria.

The present inventors through a review of available literature believe that the existing therapies and dosage regimen are not sufficient for treatment of resistant malaria and prevention of further development of parasite resistance, and, therefore, there is an immediate need for a fixed dose combination which can provide a new dosage regimen for similar and effective exposure of anti-malarial drugs across all patient groups both against sensitive and resistant malaria as well.

### SUMMARY

The present inventors have found that a body weight-based dosing regimen endows a better alternative therapy in malaria control with a potential role in both treatment of resistant malaria and prevention of the further development of parasite resistance. Such dosing regimen provided with a body weight-based fixed dose combination composition that is advantageous over existing treatments as it provides optimum dosing while avoiding sub-therapeutic dosing or over-dosing of a patient.

An aspect of the present disclosure is a body weight based dosing regimen with a fixed dose combination composition for effective exposure of anti-malarial drugs in all patient groups both against sensitive and resistant malaria.

Some exemplary embodiments of the body weight based dosing regimens with fixed dose combination compositions of the present invention are provided below:

A body weight-based dosing regimen comprising a therapeutically effective amount of Arterolane and Piperaquine for use in treating malaria, wherein the dose of Arterolane ranges from about 3 mg/kg/day to about 6.5 mg/kg/day and the dose of Piperaquine ranges from about 15 mg/kg to about 32 mg/kg/day.

A method for treating malaria in a patient comprising administering to the patient a therapeutically effective amount of Arterolane and Piperaquine in accordance with a body weight-based dosing regimen, wherein the dose of Arterolane ranges from about 3 mg/kg/day to about 6.5 mg/kg/day and the dose of Piperaquine ranges from about 15 mg/kg to about 32 mg/kg/day.

A use of Arterolane and Piperaquine in the preparation of a medicament for treating malaria, wherein a therapeutically effective amount of Arterolane and Piperaquine is administered to a patient in accordance with a body weight-based dosing regimen, wherein the dose of Arterolane ranges from about 3 mg/kg to about 6.5 mg/kg and the dose of Piperaquine ranges from about 15 mg/kg to about 32 mg/kg.

A pharmaceutical kit comprising: (i) Arterolane, (ii) Piperaquine, and (iii) instructions for administering a therapeutically effective amount of Arterolane and Piperaquine in accordance with a body weight-based dosing regimen, wherein the dose of Arterolane ranges from about 3 mg/kg/day to about 6.5 mg/kg/day and the dose of Piperaquine ranges from about 15 mg/kg to about 32 mg/kg/day.

In further enhancements, the effective amount is administered in a fixed dose pharmaceutical composition in accordance with the body weight of a patient.

In still further enhancements, the effective amount of Arterolane ranges from about 30 mg to about 350 mg and the amount of Piperaquine ranges from about 150 mg to about 2000 mg.

In still further enhancements, the amount of Arterolane ranges from about 30 mg to about 50 mg and the amount of Piperaquine ranges from about 150 mg to about 250 mg in accordance with the body weight of a patient that ranges from 5 kg to up to 11 kg.

In other alternate enhancements, the amount of Arterolane ranges from about 60 mg to about 100 mg and the amount of Piperaquine ranges from about 300 mg to about 500 mg in accordance with the body weight of a patient that ranges from 11 kg to up to 25 kg.

In yet other alternate enhancements, the amount of Arterolane ranges from about 120 mg to about 200 mg and the amount of Piperaquine ranges from about 600 mg to about 1000 mg in accordance with the body weight of a patient that ranges from 25 kg to up to 60 kg.

In still other alternate enhancements, the amount of Arterolane ranges from about 250 mg to about 350 mg and the amount of Piperaquine ranges from about 1275 mg to about 2000 mg in accordance with the body weight of a patient that ranges from 60 kg to above 80 kg.

In other enhancements, the dosing regimen comprises administering to a patient Arterolane at about 30 mg to about 50 mg and Piperaquine at about 150 mg to about 250 mg once-a-day for three days.

In further enhancements, the patient has a body weight of 5 kg to up to 11 kg.

In additional enhancements, the dosing regimen comprises administering to a patient Arterolane at about 60 mg to about 100 mg and Piperaquine at about 300 mg to about 500 mg once-a-day for three days.

In other enhancements, the patient has the body weight of 11 kg to up to 25 kg.

In alternate enhancements, the dosing regimen comprises administering to a patient Arterolane at about 120 mg to about 200 mg and Piperaquine at about 600 mg to about 1000 mg once-a-day for three days.

In other enhancements, the patient has a body weight of 25 kg to up to 60 kg.

In alternate enhancements, the dosing regimen comprises administering to a patient Arterolane at about 250 mg to about 350 mg and Piperaquine at about 1275 mg to about 2000 mg once-a-day for an at least three days.

In other enhancements, the patient has a body weight of 60 kg to above 80 kg.

In yet another enhancement, the fixed dose pharmaceutical composition containing about 32 mg of Arterolane and about 160 mg of Piperaquine, wherein the composition is administered to a patient once-a-day for three days when the body weight of the patient ranges from 5 kg to up to 8 kg.

In another alternate enhancement, the fixed dose pharmaceutical composition containing about 48 mg of Arterolane and about 240 mg of Piperaquine, wherein the composition is administered to a patient once-a-day for three days when the body weight of the patient ranges from 8 kg to up to 11 kg.

More specifically, the fixed dose pharmaceutical composition containing about 64 mg of Arterolane and about 320 mg of Piperaquine, wherein the composition is administered to a patient once-a-day for three days when the body weight of the patient ranges from 11 kg to up to 17 kg.

Alternately, the fixed dose pharmaceutical composition containing about 96 mg of Arterolane and about 480 mg of Piperaquine, wherein the composition is administered to a patient once-a-day for three days when the body weight of the patient ranges from 17 kg to up to 25 kg.

Alternately, the fixed dose pharmaceutical composition containing about 128 mg of Arterolane and about 640 mg of Piperaquine, wherein the composition is administered to a patient once-a-day for three days when the body weight of the patient ranges from 25 kg to up to 36 kg.

Still alternately, the fixed dose pharmaceutical composition containing about 192 mg of Arterolane and about 960 mg of Piperaquine, wherein the composition is administered to a patient once-a-day for three days when the body weight of the patient ranges from 36 kg to up to 60 kg.

Still alternately, the fixed dose pharmaceutical composition containing about 256 mg of Arterolane and about 1280 mg of Piperaquine, wherein the composition is administered to the patient once-a-day for three days when the body weight of the patient ranges from 60 kg to up to 80 kg.

Still alternately, the fixed dose pharmaceutical composition containing about 320 mg of Arterolane and about 2000 mg of Piperaquine, wherein the composition is administered to the patient once-a-day for three days when the body weight of the patient is 80 kg and above.

In another aspect of the disclosure, the composition is a solid oral dosage form, a liquid oral dosage form, or an intravenous/parenteral dosage form.

In yet another aspect of the disclosure, the combination is administered in conjunction with at least one additional therapeutically effective anti-malarial compound.

More specifically the anti-malarial compound is selected from Mefloquine or Primaquine.

Even more specifically the compound is administered simultaneously, concurrently or concomitantly.

The aforementioned aspects and embodiments, and other aspects, objects, features and advantages of the present invention will be apparent from the following detailed description.

### BRIEF DESCRIPTION OF THE DRAWINGS

FIG. 1: Kaplan-Meier estimates are shown for the time to P. falciparum recrudescent infections following treatment with artemether-lumefantrine (AL), arterolane-piperaquine+mefloquine (ART-PPQ+MQ) and arterolane-piperaquine (ART-PPQ).

### DETAILED DESCRIPTION

As used herein the following definitions apply unless clearly indicated otherwise. It should be understood that unless expressly stated to the contrary, the singular forms “a”, “an” and “the” include plural reference unless the context clearly dictates otherwise.

The present disclosure is based on the unexpected findings that administration of Arterolane and Piperaquine in a body weight-based dosing to a patient for the treatment of malaria provides an optimum dosing; avoids sub-therapeutic dosing or over-dosing to patients and ensures similar exposure across all patient groups. It is important to achieve effective antimalarial drug concentrations for a sufficient time (exposure) in all target populations in order to ensure high cure rates. Some patient groups, notably younger children, are not dosed optimally with the recommended flat-dosage regimen for Arterolane and Piperaquine, which compromises efficacy and fuels resistance. The unexpected absence of any variation in the exposure of drugs across all patient groups is important for clinical applications.

Accordingly, in an aspect of the present disclosure, there is provided a body weight-based dosing regimen comprising a therapeutically effective amount of Arterolane and Piperaquine for use in the treatment of malaria, wherein the dose of Arterolane ranges from about 3 mg/kg/day to about 6.5 mg/kg/day and the dose of Piperaquine ranges from about 15 mg/kg/day to about 32 mg/kg/day.

Accordingly, in another aspect of the present disclosure, there is provided a body weight-based fixed dose combination composition comprising a therapeutically effective amount of Arterolane and Piperaquine for use in the treatment of malaria, wherein the dose of Arterolane ranges from about 3 mg/kg/day to about 6.5 mg/kg/day and the dose of Piperaquine ranges from about 15 mg/kg/day to about 32 mg/kg/day.

The term “Arterolane”, as used herein, refers to Arterolane and pharmaceutically acceptable prodrugs thereof, pharmaceutically acceptable salts, pharmaceutically acceptable solvates, pharmaceutically acceptable hydrates, pharmaceutically acceptable esters, pharmaceutically acceptable enantiomers, pharmaceutically acceptable derivatives, pharmaceutically acceptable polymorphs, pharmaceutically acceptable complexes etc. Preferably, Arterolane is present as Arterolane maleate.

Arterolane is one of first fully synthetic trioxolane peroxide, non-artemisinin antimalarial compound. The molecular formula is C₂₆H₄₀N₂O₈ and molecular weight is 508.61. The Structural formula as shown below:

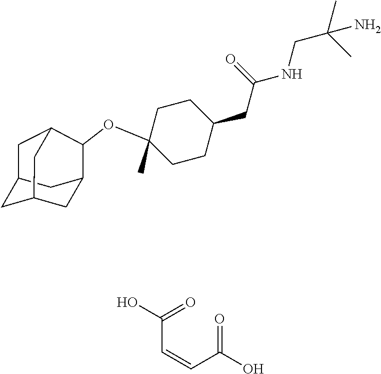 [SMILES: CC(C)(N)CNC(=O)C[C@H]1CC[C@](C)(OC2C3CC4CC(C3)CC2C4)CC1 ; O=C(O)/C=C\C(=O)O]

It has rapid schizontocidal activity against all erythrocytic stages of P. falciparum without any effect on hepatic stages. This action of Arterolane is attributed to inhibition of heme detoxification and Pf-encoded sarcoplasmic endoplasmic reticulum calcium ATPase (PfATP6). Arterolane is an active moiety, which gets accumulated either in cytosol or food vacuole of the parasite.

The term “Piperaquine”, as used herein, refers to Piperaquine and pharmaceutically acceptable prodrugs thereof, pharmaceutically acceptable salts, pharmaceutically acceptable solvates, pharmaceutically acceptable hydrates, pharmaceutically acceptable esters, pharmaceutically acceptable enantiomers, pharmaceutically acceptable derivatives, pharmaceutically acceptable polymorphs, pharmaceutically acceptable complexes etc. Preferably, Piperaquine is present as Piperaquine phosphate.

Piperaquine, a synthetic bisquinoline compound belonging to 4-amioquinoline group of antimalarials. The molecular formula of Piperaquine phosphate is C₂₉H₃₂Cl₂N₆.4H₃PO₄.4H₂O and molecular weight is 999.56. The structural formula is given below:

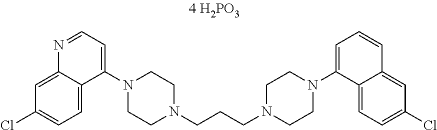 [SMILES: Clc1ccc2c(N3CCN(CCCN4CCN(c5ccnc6cc(Cl)ccc56)CC4)CC3)cccc2c1]

Piperaquine has a slow and longer schizontocidal activity against erythrocytic stages of both P. vivax and P. falciparum and Chloroquine-resistant plasmodium strains. Most evidences conclusively propose inhibition of parasite heme digestion pathway, similar to action of Chloroquine.

As used herein the term, “therapeutically effective amount” refers to an amount sufficient to provide a therapeutic benefit for the treatment or management of the malaria.

As used herein, the term “about”, refers to any value which lies within the range defined by a variation of up to ±10% of the value.

In one embodiment, Arterolane is administered at a weight-based dose ranging from about 3 mg/kg/day to 7 mg/kg/day.

In another embodiment, Arterolane is administered to a malaria patient at a weight-based dose ranging from about 3 mg/kg to about 6.5 mg/kg, about 3.2 mg/kg to about 6.5 mg/kg, about 3.5 mg/day to about 6.5 mg/kg, about 3.7 mg/kg to about 6.5 mg/kg, about 4.0 mg/kg to about 6.5 mg/kg, about 4.3 mg/kg to about 6.5 mg/kg, about 3 mg/kg to about 5.5 mg/kg, about 3.2 mg/kg to about 5.5 mg/kg, about 3.5 mg/day to about 5.5 mg/kg, about 3.7 mg/kg to about 5.5 mg/kg, about 4.0 mg/kg to about 5.5 mg/kg, about 4.3 mg/kg to about 5.5 mg/kg, about 3.2 mg/kg to about 6.4 mg/kg, about 3.56 to about 6.4 mg/kg, about 3.84 mg/kg to about 6.4 mg/kg, about 3.76 mg/kg to about 6.4 mg/kg, about 4 mg/kg to about 6.4 mg/kg, about 4.36 mg/kg to about 6.4 mg/kg, about 5.12 mg/kg to about 6.4 mg/kg, about 5.33 mg/kg to about 6.4 mg/kg, about 5.64 mg/kg to about 6.4 mg/kg, about 5.81 mg/kg to about 6.4 mg/kg or about 6.0 mg/kg to about 6.4 mg/kg.

In a preferred embodiment, Arterolane is administered to a malaria patient at a weight-based dose ranging from about 3.2 mg/kg to about 6.4 mg/kg, about 3.56 mg/kg to about 6.4 mg/kg, about 3.84 mg/kg to about 6.4 mg/kg, about 3.76 mg/kg to about 6.4 mg/kg, about 4 mg/kg to about 6.4 mg/kg, about 4.36 mg/kg to about 6.4 mg/kg, about 5.12 mg/kg to about 6.4 mg/kg, about 5.33 mg/kg to about 6.4 mg/kg, about 5.64 mg/kg to about 6.4 mg/kg, about 5.81 mg/kg to about 6.4 mg/kg or about 6.0 mg/kg to about 6.4 mg/kg.

In one embodiment, Piperaquine is administered to a malaria patient at a weight-based dose ranging from about 15 mg/kg/day to about 32.0 mg/kg/day.

In another embodiment, Piperaquine is administered to a malaria patient at a weight-based dose ranging from about 15 mg/kg to about 35 mg/kg, 16 mg/kg to about 35 mg/kg, about 17 mg/kg to about 35 mg/kg, about 17.7 mg/day to about 35 mg/kg, about 18 mg/kg to about 35 mg/kg, about 18.75 mg/kg to about 35 mg/kg, about 19 mg/kg to about 35 mg/kg, about 19.2 mg/kg to about 35 mg/kg, about 20 mg/kg to about 35 mg/kg, about 20.5 mg/day to about 35 mg/kg, about 21 mg/kg to about 35 mg/kg, about 21.75 mg/kg to about 35 mg/kg, about 16 mg/kg to about 32 mg/kg, about 17 mg/kg to about 32 mg/kg, about 17.7 mg/day to about 32 mg/kg, about 18 mg/kg to about 32 mg/kg, about 18.75 mg/kg to about 32 mg/kg, about 19 mg/kg to about 32 mg/kg, about 19.2 mg/kg to about 32 mg/kg, about 20 mg/kg to about 32 mg/kg, about 20.5 mg/day to about 32 mg/kg, about 21 mg/kg to about 32 mg/kg, about 21.75 mg/kg to about 32 mg/kg, 16 mg/kg to about 32 mg/kg, about 17.78 mg/kg to about 32 mg/kg, about 18.82 mg/day to about 32 mg/kg, about 19.2 mg/kg to about 32 mg/kg, about 20 mg/kg to about 32 mg/kg, about 21.33 mg/kg to about 32 mg/kg, about 21.82 mg/kg to about 32 mg/kg, about 25.6 mg/kg to about 32 mg/kg, about 26.66 mg/day to about 32 mg/kg, about 28.23 mg/kg to about 32 mg/kg, about 29.09 mg/kg to about 32 mg/kg or about 30.0 mg/kg to about 32 mg/kg.

In a preferred embodiment, Piperaquine is administered to a malaria patient at a weight-based dose ranging from about 16 mg/kg to about 32 mg/kg, about 17.78 mg/kg to about 32 mg/kg, about 18.82 mg/day to about 32 mg/kg, about 19.2 mg/kg to about 32 mg/kg, about 20 mg/kg to about 32 mg/kg, about 21.33 mg/kg to about 32 mg/kg, about 21.82 mg/kg to about 32 mg/kg, about 25.6 mg/kg to about 32 mg/kg, about 26.66 mg/day to about 32 mg/kg, about 28.23 mg/kg to about 32 mg/kg, about 29.09 mg/kg to about 32 mg/kg or about 30.0 mg/kg to about 32 mg/kg.

In one embodiment, the body weight of the patient is from about 5 kg to about 100 kg. In another embodiment, the body weight of the patient is about 5 kg to about 80 kg, about 11 kg to about 80 kg, about 17 kg to about 80 kg, about 25 kg to about 80 kg, about 36 kg to about 80 kg, about 60 kg to about 80 kg, about 5 kg to about 60 kg, about 11 kg to about 60 kg, about 17 kg to about 60 kg, about 25 kg to about 60 kg, about 36 kg to about 60 kg, about 5 kg to about 8 kg, 5 kg to about 11 kg, 8 kg to about 11 kg, about 11 kg to 17 kg, about 11 kg to about 25 kg, about 17 kg to about 25 kg, about 25 kg to 36 kg, 5 kg to 7.9 kg, 8 kg to 10.9 kg, 11 kg to 16.9 kg, 17 kg to 24.9 kg, 25 kg to 35.9 kg, 36 kg to 59.9 kg or 60 kg to 79.9 kg.

In a preferred embodiment, the body weight of the patient is from 5 kg to 8 kg, 5 kg to 11 kg, 8 kg to 11 kg, 11 kg to 17 kg, 11 kg to 25 kg, 17 kg to 25 kg, 25 kg to 36 kg, 36 kg to 60 kg, 60 kg to 80 kg or above 80 kg.

In a preferred embodiment, the body weight of the patient is from 5 kg to 7.9 kg, 8 kg to 10.9 kg, 11 kg to 16.9 kg, 17 kg to 24.9 kg, 25 kg to 35.9 kg, 36 kg to 59.9 kg, 60 kg to 79.9 kg or 80 kg and above.

In a preferred embodiment, the dosage of Arterolane and Piperaquine is administered once-a-day for a 3 days.

In another embodiment, about 30 mg to about 50 mg of Arterolane and about 100 mg to about 250 mg of Piperaquine are administered to a patient once-a-day for three days when the body weight of the patient is 5 kg to up to 11 kg.

In another embodiment, about 60 mg to about 100 mg of Arterolane and about 300 mg to about 500 mg of Piperaquine are administered to a patient once-a-day for three days when the body weight of the patient is 11 kg to up to 25 kg.

In another embodiment, about 120 mg to about 200 mg of Arterolane and about 600 mg to about 1000 mg of Piperaquine are administered to a patient once-a-day for three days when the body weight of the patient is 25k g to up to 60 kg.

In yet another embodiment, about 240 mg to about 320 mg of Arterolane and about 1080 mg to about 2000 mg of Piperaquine are administered to a patient once-a-day for three days when the body weight of the patient is 60 kg to above 80 kg.

In a preferred embodiment, about 32 mg of Arterolane and about 160 mg of Piperaquine are administered to a patient once-a-day for three days when the body weight of the patient is 5 kg to up to 8 kg.

In a preferred embodiment, about 48 mg of Arterolane and about 240 mg of Piperaquine are administered to a patient once-a-day for three days when the body weight of the patient is 8 kg to up to 11 kg.

In another preferred embodiment, about 64 mg of Arterolane and about 320 mg of Piperaquine are administered to a patient once-a-day for three days when the body weight of the patient is 11 kg to up to 17 kg.

In another preferred embodiment, about 96 mg of Arterolane and about 480 mg of Piperaquine are administered to a patient once-a-day for three days when the body weight of the patient is 17 kg to up to 25 kg.

In another preferred embodiment, about 128 mg of Arterolane and about 640 mg of Piperaquine are administered to a patient once-a-day for three days when the body weight of the patient is 25 kg to up to 36 kg.

In yet another preferred embodiment, about 192 mg of Arterolane and about 960 mg of Piperaquine are administered to a patient once-a-day for three days when the body weight of the patient is 36 kg to up to 60 kg.

In yet another preferred embodiment, about 256 mg of Arterolane and about 1280 mg of Piperaquine are administered to a patient once-a-day for three days when the body weight of the patient is 60 kg to up to 80 kg.

In yet another preferred embodiment, about 320 mg of Arterolane and about 1600 mg of Piperaquine are administered to a patient once-a-day for three days when the body weight of the patient is 80 kg and above.

In a preferred embodiment, the present invention provides a dosing regimen or a fix dose combination composition for the administration of Arterolane and Piperaquine to a patient for the treatment of malaria, comprising administering to a patient with a:
body weight of 5 kg to up to 11 kg, a daily dose of about 30 mg to about 50 mg of Arterolane and about 100 mg to about 250 mg of Piperaquine,
body weight of 11 kg to up to 25 kg, a daily dose of about 60 mg to about 100 mg of Arterolane and about 300 mg to about 500 mg of Piperaquine,
body weight of 25 kg to up to 60 kg, a daily dose of about 120 mg to about 200 mg of Arterolane and about 600 mg to about 1000 mg of Piperaquine, or
body weight of 60 kg to above 80 kg, a daily dose of about 240 mg to about 320 mg of Arterolane and about 1080 mg to about 2000 mg of Piperaquine.

In yet another preferred embodiment, the present invention provides a dosing regimen or a fixed dose combination composition for the administration of Arterolane and Piperaquine to a patient for the treatment of malaria, comprising administering to a patient with a:
body weight of 5 kg to up to 8 kg, a daily dose of about 32 mg of Arterolane and about 160 mg of Piperaquine,
body weight of 8 kg to up to 11 kg, a daily dose of about 48 mg of Arterolane and about 240 mg of Piperaquine,
body weight of 11 kg to up to 17 kg, a daily dose of about 64 mg of Arterolane and about 320 mg of Piperaquine,
body weight of 17 kg to up to 25 kg, a daily dose of about 96 mg of Arterolane and about 480 mg of Piperaquine,
body weight of 25 kg to up to 36 kg, a daily dose of about 128 mg of Arterolane and about 640 mg of Piperaquine,
body weight of 36 kg to up to 60 kg, a daily dose of about 192 mg of Arterolane and about 960 mg of Piperaquine,
body weight of 60 kg to up to 80 kg, a daily dose of about 256 mg of Arterolane and about 1280 mg of Piperaquine, or
body weight of 80 kg and above, a daily dose of about 320 mg of Arterolane and about 1600 mg of Piperaquine.

As described herein, a body weight-based dosing regimen has therapeutic applications and may be used to effectively treat acute uncomplicated P. falciparum malaria.

Thus, the present invention in an aspect provides a method for treating malaria in a patient comprising administering to the patient a therapeutically effective amount of Arterolane and Piperaquine in accordance with a body weight-based dosing regimen, wherein the dose of Arterolane ranges from about 3 mg/kg/day to about 6.5 mg/kg/day and the dose of Piperaquine ranges from about 15 mg/kg to about 32 mg/kg/day.

In another embodiment, the present invention provides use of a fixed dose combination composition of Arterolane and Piperaquine in a method for treating malaria in a patient comprising administering to the patient a therapeutically effective amount of Arterolane and Piperaquine in accordance with a body weight-based dosing regimen, wherein the dose of Arterolane ranges from about 3 mg/kg/day to about 6.5 mg/kg/day and the dose of Piperaquine from about 15 mg/kg to about 32 mg/kg/day.

As used herein the term “Treatment” or “Treating” refers to cure the infection as rapidly as possible and to prevent the progression to severe disease. The term “cure” refers to elimination of all parasites from the body. The treatment of resistant malaria and prevention of further development of parasite resistance is also envisioned by the present invention.

As used herein the term “patient” refers to a subject such as human suffering from malaria infection as defined hereinbefore and needs therapeutic intervention for the treatment of such infections. The patient includes, for example, children, adults or the elderly.

According to another aspect, there is provided a use of Arterolane and Piperaquine in the preparation of a medicament for treating malaria, wherein a therapeutically effective amount of Arterolane and Piperaquine is administered to a patient in accordance with a body weight-based dosing regimen, wherein the dose of Arterolane ranges from about 3 mg/kg to about 6.5 mg/kg and the dose of Piperaquine from about 15 mg/kg to about 30 mg/kg.

As used herein the phrase “preparation of a medicament” includes the use of the dosing regimen directly as the medicament in addition to their use in any stage of the preparation of such a medicament.

According to another aspect of the present disclosure, there is provided a fixed dose pharmaceutical composition comprising a therapeutically effective amount of Arterolane and Piperaquine together with pharmaceutically acceptable excipients.

In a preferred embodiment, the present disclosure fixed dose pharmaceutical composition of Arterolane and Piperaquine is meant for oral administration. A composition suitable for oral use includes dosage forms, but not limited, to tablets, capsules, dispersible tablets, sachets or sprinkles. In a particular embodiment, the solid dosage form is in the form of tablets or dispersible tablets. If the solid dosage form is a tablet, the tablet can be of any suitable shape such as round, spherical, or oval. The tablet may have a monolithic or a multi-layered structure.

The fixed dose pharmaceutical composition of the present disclosure can be obtained by conventional approaches using conventional pharmaceutically acceptable excipients well known in the art. Examples of pharmaceutically acceptable excipients suitable for tablet preparation include, but not limited to, diluents selected from a group comprising of calcium phosphate-dibasic, calcium carbonate, lactose, glucose, microcrystalline cellulose, cellulose powdered, silicified microcrystalline cellulose, calcium silicate, starch, starch pregelatinized, or polyols such as mannitol, sorbitol, xylitol, maltitol, and sucrose or combinations thereof; binders selected from a group comprising of starch, pre-gelatinized starch, carboxymethyl cellulose, sodium cellulose, microcrystalline cellulose, hydroxyproyl cellulose, hydroxypropyl methylcellulose, polyvinylpyrrolidone, crospovidone, or combinations thereof; disintegrants selected from a group comprising of crosslinked cellulose, crosslinked-polyvinylpyrrolidone (crosspovidone), sodium starch glycolate, polyvinylpyrrolidone (polyvidone, povidone), sodium carboxymethylcellulose, cross-linked sodium carboxymethylcellulose (croscarmellose sodium), hydroxypropyl cellulose, hydroxypropyl methylcellulose, xanthan gum, alginic acid, or soy polysaccharides or combinations thereof; wetting agents selected from a group comprising of polysorbate, sodium lauryl sulphate, or glyceryl stearate or combinations thereof; or lubricants selected from a group comprising of sodium lauryl sulfate, talc, magnesium stearate, sodium stearyl fumarate, stearic acid, glyceryl behenate, hydrogenated vegetable oil, or zinc stearate or combinations thereof.

In a further embodiment, the tablets prepared in the present disclosure may be uncoated or coated for altering their disintegration, and subsequent enteral absorption of the active ingredient, or for improving their stability and/or appearance. In both cases, conventional coating agents and approaches well known in the art can be employed.

According to another embodiment of the present disclosure, the therapeutically effective amount of Arterolane and Piperaquine is administered in conjunction with at least one additional therapeutically effective anti-malarial compound. Preferably, the additional anti-malarial compound is Mefloquine or Primaquine. It is believed the use of such a triple combination with different modes of action has the potential to reduce emergence of drug resistance.

In yet another embodiment, the therapeutically effective anti-malarial compound is administered simultaneously, concurrently or concomitantly with the dosing regimen of the present invention.

In another embodiment, the present disclosure also provides a dosing regimen for the treatment of malaria comprising administering Primaquine or Mefloquine in fixed dose combination with Arterolane and Piperaquine in a body weight based dosing, wherein Arterolane and Piperaquine are administered at a dose of about 3.0 mg/kg/day to about 6.5 mg/kg/day and about 16.0 mg/kg/day to about 32.0 mg/kg/day, respectively, to a patient weighing 5 kg to 100 kg. In a preferred embodiment, Arterolane and Piperaquine are administered at a dose of about 4.1 mg/kg/day to about 5.5 mg/kg/day and about 18.0 mg/kg/day to about 30.0 mg/kg/day, respectively, to a patient weighing 5 kg to 100 kg.

In a preferred embodiment of the present disclosure, Mefloquine is administered at a dose of about 5 mg/kg/day to about 15 mg/kg/day, preferably about 5.7 mg/kg/day to about 11.4 mg/kg/day, once-a-day for three days. In another preferred embodiment, Primaquine is administered at a dose of about 0.1 mg/kg/day to about 1 mg/kg/day, preferably, about 0.15 mg/kg/day to about 0.4 mg/kg/day.

In another embodiment, the present disclosure provides a method of treatment of malaria comprising administering a triple combination of Arterolane, Piperaquine and Mefloquine, wherein the Arterolane and Piperaquine are administered to a patient in a body weight based dosing, wherein Arterolane and Piperaquine are administered at a dose of about 3.0 mg/kg/day to about 6.5 mg/kg/day and about 16.0 mg/kg/day to about 32.0 mg/kg/day, respectively, to the patient weighing 5 kg to 100 kg. In a preferred embodiment, Arterolane and Piperaquine are administered at a dose of about 4.1 mg/kg/day to about 5.5 mg/kg/day and about 18.0 mg/kg/day to about 30.0 mg/kg/day, respectively, to a patient weighing 5 kg to 100 kg.

In a preferred embodiment, Mefloquine is administered at a dose of 5 mg/kg/day to 15 mg/kg/day, preferably about 5.7 mg/kg/day to about 11.4 mg/kg/day, wherein the combined dosage of Arterolane, Piperaquine and Mefloquine is administered to a patient once-a-day for three days. In a preferred embodiment, a single dose of Primaquine is administered to a malaria patient in combination with Arterolane, Piperaquine and Mefloquine.

In another related embodiment, Arterolane and Piperaquine are administered at a dose of about 3.0 mg/kg/day to about 6.5 mg/kg/day and about 16.0 mg/kg/day to about 32.0 mg/kg/day, respectively, to a patient weighing 5 kg to 100 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In yet another embodiment, Arterolane and Piperaquine are administered at a dose of about 4.1 mg/kg/day to about 5.5 mg/kg/day and about 18.0 mg/kg/day to about 30.0 mg/kg/day, respectively, to a patient weighing 5 kg to 100 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In another embodiment, about 30 mg to about 50 mg of Arterolane and about 100 mg to about 250 mg of Piperaquine are administered to a patient weighing 5 kg to up to 11 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In yet another embodiment, about 60 mg to about 100 mg of Arterolane and about 300 mg to about 500 mg of Piperaquine are administered to a patient weighing 11 kg to up to 25 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In another embodiment, about 120 mg to about 200 mg of Arterolane and about 600 mg to about 1000 mg of Piperaquine are administered to a patient weighing 25 kg to up to 60 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to 0.4 mg/kg/day.

In yet another embodiment, about 240 mg to about 320 mg of Arterolane and about 1080 mg to about 2000 mg of Piperaquine are administered to a patient weighing 60 kg to above 80 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In a preferred embodiment, about 32 mg of Arterolane and about 160 mg of Piperaquine are administered to a patient weighing 5 kg to up to 8 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In a preferred embodiment, about 48 mg of Arterolane and about 240 mg of Piperaquine are administered to a patient weighing 8 kg to up to 11 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In a preferred embodiment, about 64 mg of Arterolane and about 320 mg of Piperaquine are administered to a patient weighing 11 kg to up to 17 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In a preferred embodiment, about 96 mg of Arterolane and about 480 mg of Piperaquine are administered to a patient weighing 17 kg to up to 25 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In a preferred embodiment, about 128 mg of Arterolane and about 640 mg of Piperaquine are administered to a patient weighing 25 kg to up to 36 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of

Primaquine may be administered at a dose of about 0.1 mg/kg/day to 1 mg/kg/day, preferably, about 0.15 mg/kg/day to about 0.4 mg/kg/day.

In a preferred embodiment, about 192 mg of Arterolane and about 960 mg of Piperaquine are administered to a patient weighing 36 kg to up to 60 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In a preferred embodiment, about 256 mg of Arterolane and about 1280 mg of Piperaquine are administered to a patient weighing 60 kg to up to 80 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In a preferred embodiment, about 320 mg of Arterolane and about 1600 mg of Piperaquine are administered to a patient weighing above 80 kg, and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In a further embodiment, the fixed dose combination composition comprising a therapeutically effective amount of Arterolane and Piperaquine according to present invention is a solid oral dosage form, liquid oral dosage form or an intravenous/parenteral dosage form.

In yet another preferred embodiment, there is provided a method of treating malaria comprising administering a triple combination of Arterolane, Piperaquine and Mefloquine, wherein Arterolane and Piperaquine are administered in a dosing regimen comprising administering to a patient with a:
body weight of 5 kg to up to 11 kg, a daily dose of about 30 mg to about 50 mg of Arterolane and about 100 mg to about 250 mg of Piperaquine,
body weight of 11 kg to up to 25 kg, a daily dose of about 60 mg to about 100 mg of Arterolane and about 300 mg to about 500 mg of Piperaquine,
body weight of 25 kg to up to 60 kg, a daily dose of about 120 mg to about 200 mg of Arterolane and about 600 mg to about 1000 mg of Piperaquine, or
body weight of 60 kg to above 80 kg, a daily dose of about 240 mg to about 320 mg of Arterolane and about 1080 mg to about 2000 mg of Piperaquine,
   and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In yet another preferred embodiment, there is provided a method of treating malaria comprising administering to a patient a combination of Arterolane, Piperaquine and Mefloquine, wherein Arterolane and Piperaquine are administered in a dosing regimen comprising administering to a patient with a:
body weight of 5 kg to up to 8 kg, a daily dose of about 32 mg of Arterolane and about 160 mg of Piperaquine,
body weight of 8 kg to up to 11 kg, a daily dose of about 48 mg of Arterolane and about 240 mg of Piperaquine,
body weight of 11 kg to up to 17 kg, a daily dose of about 64 mg of Arterolane and about 320 mg of Piperaquine,
body weight of 17 kg to up to 25 kg, a daily dose of about 96 mg of Arterolane and about 480 mg of Piperaquine,
body weight of 25 kg to up to 36 kg, a daily dose of about 128 mg of Arterolane and about 640 mg of Piperaquine,
body weight of 36 kg to up to 60 kg, a daily dose of about 192 mg of Arterolane and about 960 mg of Piperaquine,
body weight of 60 kg to up to 80 kg, a daily dose of about 256 mg of Arterolane and about 1280 mg of Piperaquine, or
body weight of 80 kg and above, a daily dose of about 320 mg of Arterolane and about 1600 mg of Piperaquine,
   and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In yet another preferred embodiment, there is provided a use of a triple combination of Arterolane, Piperaquine and Mefloquine in a method of treating malaria by administering the combination, wherein Arterolane and Piperaquine are administered in a dosing regimen comprising administering to a patient with a:
body weight of 5 kg to up to 11 kg, a daily dose of about 30 mg to about 50 mg of Arterolane and about 100 mg to about 250 mg of Piperaquine,
body weight of 11 kg to up to 25 kg, a daily dose of about 60 mg to about 100 mg of Arterolane and about 300 mg to about 500 mg of Piperaquine,
body weight of 25 kg to up to 60 kg, a daily dose of about 120 mg to about 200 mg of Arterolane and about 600 mg to about 1000 mg of Piperaquine, or
body weight of 60 kg to above 80 kg, a daily dose of about 240 mg to about 320 mg of Arterolane and about 1080 mg to about 2000 mg of Piperaquine,
   and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In yet another preferred embodiment, there is provided a use of a triple combination of Arterolane, Piperaquine and Mefloquine in a method of treating malaria by administering the combination, wherein Arterolane and Piperaquine are administered in a dosing regimen comprising administering to a patient with a:
body weight of 5 kg to up to 8 kg, a daily dose of about 32 mg of Arterolane and about 160 mg of Piperaquine,
body weight of 8 kg to up to 11 kg, a daily dose of about 48 mg of Arterolane and about 240 mg of Piperaquine,
body weight of 11 kg to up to 17 kg, a daily dose of about 64 mg of Arterolane and about 320 mg of Piperaquine,
body weight of 17 kg to up to 25 kg, a daily dose of about 96 mg of Arterolane and about 480 mg of Piperaquine,
body weight of 25 kg to up to 36 kg, a daily dose of about 128 mg of Arterolane and about 640 mg of Piperaquine,
body weight of 36 kg to up to 60 kg, a daily dose of about 192 mg of Arterolane and about 960 mg of Piperaquine,
body weight of 60 kg to up to 80 kg, a daily dose of about 256 mg of Arterolane and about 1280 mg of Piperaquine, or
body weight of 80 kg and above, a daily dose of about 320 mg of Arterolane and about 1600 mg of Piperaquine,
   and Mefloquine is administered at a dose of about 5 mg/kg/day to about 15 mg/kg/day, preferably about 5.7 mg/kg/day to about 11.4 mg/kg/day, once-a-day for three days. Optionally, a single dose of Primaquine may be administered at a dose of about 0.1 mg/kg/day to about 1 mg/kg/day, preferably, about 0.15 mg/kg/day to about 0.4 mg/kg/day.

In another embodiment, Mefloquine is administered as an oral dosage form. Preferably, the oral dosage form is in the form of tablet, capsule, dispersible tablets, sachets, sprinkles, liquids, solution, suspension or emulsion. More preferably, the oral dosage form is in the form of tablet or solution. In an aspect of the present invention, the oral dosage form comprising Mefloquine is in the form of a tablet comprising about 250 mg of Mefloquine. In yet another aspect, the oral dosage form comprising Mefloquine is in the form of a solution comprising about 50 mg/mL of Mefloquine. In a further aspect, a suspension of Mefloquine is made by allowing one tablet (250 mg) to dissolve in 5 mL of water or other beverage such as sweet juices.

In another embodiment, the present invention provides a pharmaceutical kit comprising:
(i) Arterolane;
(ii) Piperaquine, and
(iii) instructions for administering a therapeutically effective amount of Arterolane and Piperaquine in accordance with a body weight-based dosing regimen,
wherein the dose of Arterolane ranges from about 3 mg/kg/day to about 6.5 mg/kg/day and the dose of Piperaquine ranges from about 15 mg/kg to about 32 mg/kg/day.

In a further embodiment, the kit is for use in treating acute uncomplicated P. falciparum malaria. In another aspect, the present invention provides kits for the treatment of malaria comprising a fixed-dose pharmaceutical composition containing Arterolane and Piperaquine. The kit may further comprise additional anti-malarial drugs, such as Mefloquine or Primaquine.

In yet another embodiment, the kit contains tablets comprising Arterolane and Piperaquine, and tablets comprising Mefloquine.

In yet another embodiment, the kit contains tablets comprising Arterolane and Piperaquine, and a solution formulation comprising Mefloquine.

In another embodiment, the kit contains instructions for administering Arterolane and Piperaquine at a dose of about 3.0 mg/kg/day to about 6.5 mg/kg/day and about 16.0 mg/kg/day to about 32.0 mg/kg/day, respectively, to a malaria patient weighing 5 kg to 100 kg once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In yet another embodiment, the kit contains instructions for administering Arterolane and Piperaquine at a dose of about 4.1 mg/kg/day to about 5.5 mg/kg/day and about 18.0 mg/kg/day to about 30.0 mg/kg/day, respectively, to a malaria patient weighing 5 kg to 100 kg once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In a preferred embodiment, the kit contains instructions for administering Arterolane and Piperaquine at a dose of about 30 mg to about 50 mg and about 100 mg to about 250 mg, respectively, to a malaria patient weighing 5 kg to up to 11 kg. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In a further preferred embodiment, the kit contains instructions for administering Arterolane and Piperaquine at a dose of about 60 mg to about 100 mg and about 300 mg to about 500 mg, respectively, to a malaria patient weighing 11 kg to up to 25 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In yet another preferred embodiment, the kit contains instructions for administering Arterolane and Piperaquine at a dose of about 120 mg to about 200 mg and about 600 mg to about 1000 mg, respectively, to a malaria patient weighing 25 kg to up to 60 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In a further preferred embodiment, the kit contains instructions for administering Arterolane and Piperaquine at a dose of about 240 mg to about 320 mg and about 1080 mg to about 2000 mg, respectively, to a malaria patient weighing 60 kg to above 80 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In another preferred embodiment, the kit contains instructions for administering Arterolane and Piperaquine at a dose of about 32 mg and about 160 mg, respectively, to a malaria patient weighing 5 kg to up to 8 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In yet another preferred embodiment, the kit contains instructions for administering Arterolane and Piperaquine at a dose of about 48 mg and about 240 mg, respectively, to a malaria patient weighing 8 kg to up to 11 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In another preferred embodiment, the kit contains instructions for administering Arterolane and Piperaquine at a dose of about 64 mg and about 320 mg, respectively, to a malaria patient weighing 11 kg to up to 17 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In yet another preferred embodiment, the kit contains instructions for administering Arterolane and Piperaquine at a dose of about 96 mg and about 480 mg, respectively, to a malaria patient weighing 17 kg to up to 25 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In yet another preferred embodiment, the kit contains instructions for administering Arterolane and Piperaquine at a dose of about 128 mg and about 640 mg, respectively, to a malaria patient weighing 25 kg to up to 36 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In a preferred embodiment, the kit contains instructions for administering Arterolane and Piperaquine at a dose of about 192 mg and about 960 mg, respectively, to a malaria patient weighing 36 kg to up to 60 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In yet another preferred embodiment, the kit contains instructions for administering Arterolane and Piperaquine at a dose of about 256 mg and about 1280 mg, respectively, to a malaria patient weighing 60 kg to up to 80 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

In yet another preferred embodiment, the kit contains instructions for administering Arterolane and Piperaquine at a dose of about 320 mg and about 1600 mg, respectively, to a malaria patient weighing above 80 kg, once-a-day for three days. Optionally, the kit contains instructions for administering Mefloquine at a dose of about 5 mg/kg/day to about 15 mg/kg/day, preferably about 5.7 mg/kg/day to about 11.4 mg/kg/day, and a single dose of Primaquine at a dose of about 0.1 mg/kg/day to about 1 mg/kg/day, preferably, about 0.15 mg/kg/day to about 0.4 mg/kg/day.

The present disclosure is illustrated below by reference to the following examples. However, one skilled in the art will appreciate that specific methods and techniques and results discussed throughout this disclosure are merely illustrative, as innumerable variations, modifications, applications, and extensions of these embodiments and principles can be made without departing from the spirit and scope of the disclosure.

### EXAMPLES

### Example 1

An open-label randomized trial was done to assess the therapeutic efficacy and tolerability of Arterolane-Piperaquine plus single low dose of Primaquine versus Arterolane-Piperaquine plus Mefloquine and a single low dose of Primaquine versus Artemether-Lumefantrine plus a single low dose Primaquine in the treatment of uncomplicated P. falciparum malaria in children in Kenya.

In this single centre, open-label, randomized trial, 218 patients with uncomplicated Plasmodium falciparum malaria in Kilifi County Hospital in Kenya were recruited. Eligible patients were aged 2 to 12 years, with acute uncomplicated P falciparum malaria. Patients were randomly assigned (1:1:1) to either arterolane-piperaquine, arterolane-piperaquine plus mefloquine or artemether-lumefantrine. The primary endpoint was 42-day PCR-corrected efficacy. Secondary endpoints focused on the safety, tolerability and pharmacokinetics of the study drugs.

TABLE 1

| Arterolane Arm | TACT arm | Reference ACT Arm |
|---|---|---|
| Arterolane- | J Arterolane- | Artemether- |
| Piperaquine | Piperaquine x 3 days | Lumefantrine |
| x 3 days | J Mefloquine x 3 days | x3 days |

In addition, all patients will be treated at Day 0,
Hr 0 (D0H0) with a single dose of
Primaquine as a gametocidal treatment using an aged based dosing regimen

Patients were treated with body weight-based doses, for example, the dosing schedule of Arterolane maleate-Piperaquine phosphate was as shown in below Table 2:

TABLE 2

| | Arterolane- | Arterolane- | Arterolane- |
|---|---|---|---|
| | Piperaquine given | Piperaquine | Piperaquine |
| Weight (Kg) | daily for 3 days (mg) | (37.5/187.5 mg) | (150/750 mg) |
| 5-7.9 | 32 + 160 | 0.75 | 0 |
| 8-10.9 | 48 + 240 | 1.25 | 0 |
| 11-16.9 | 64 + 320 | 1.75 | 0 |
| 17-24.9 | 96 + 480 | 2.5 | 0 |
| 25-35.9 | 128 + 640 | 3.5 | 0 |
| 36-59.91 | 192 + 960 | 1 | 1 |
| 60-79.9 | 256 + 1280 | 3 | 1 |
| 80-100 | 320 + 1600 | 1 | 2 |

TABLE 3
Mefloquine dosing schedule:
Mefloquine dosing schedule (administered at HO, H24 and H48)

| Body weight (kg) | Milliliter (50 mg/ml) |
|---|---|
| 5-5.9 | 0.8 milliliter |
| 6-6.9 | 1 milliliter |
| 7-7.9 | 1.2 milliliter |
| 8-8.9 | 1.3 milliliter |
| 9-9.9 | 1.5 milliliter |
| 10-10.9 | 1.7 milliliter |
| 11-11.9 | 1.8 milliliter |
| Body weight (kg) | Tablets (250mg/tablet) |
| 12-16.9 | 0.5 tablet |
| 17-23.9 | 0.75 tablet |
| 24-33.9 | 1 tablet |
| 34-43.9 | 1.25 tablets |
| 44-48.9 | 1.5 tablets |
| 49-53.9 | 1.75 tablets |
| 54-63.9 | 2 tablets |
| 64-71.9 | 2.25 tablets |
| 72-77.9 | 2.5 tablets |
| 78-100 | 2.75 tablets |

TABLE 4: Artemether-Lumefantrine dosing schedule:
Artemether-Lumefantrine dosing schedule
(administered at H0, H8, H24, H36, H48 and H60)

| Body weight (kg) | Artemether-Lumefantrine tablet (20mg/120mg) |
|---|---|
| 5-14.9 | 1 tablet |
| 15-24.9 | 2 tablets |
| 25-34.9 | 3 tablets |
| ≥35 | 4 tablets |

TABLE 5
Primaquine dosing schedule:
Primaquine dosing schedule (administered at H24)

| Age (months) | Primaquine dose base in mg |
|---|---|
| 6-<12 | 1.25 mg |
| 12-<72 | 2.5 mg |
| 72-<120 | 5 mg |
| 120-<180 | 7.5 mg |

TABLE 6
Proposed dosing for Arterolane and Piperaquine:

| | Arterolane- | ART | ART | PQ | PQ |
|---|---|---|---|---|---|
| Body weight | Piperaquine give | Min | max | Min | Max |
| (Kg) | daily for 3 days (mg) | (mg/kg) | (mg/kg) | (mg/kg) | (mg/kg) |
| 5 to <8 | 32 + 160 | 4 | 6.4 | 20 | 32 |
| 8 to <11 | 48 + 240 | 4.4 | 6 | 21.8 | 30 |
| 11 to <17 | 64 + 320 | 3.8 | 5.8 | 18.8 | 29.1 |
| 17 to < 25 | 96 + 480 | 3.8 | 5.6 | 19.2 | 28.2 |
| 25 to <36 | 128 + 640 | 3.6 | 5.1 | 17.8 | 25.6 |
| 36 to <60 | 192 + 960 | 3.2 | 5.3 | 16 | 26.7 |
| 60<80 | 256 + 1280 | 3.2 | 4.3 | 16 | 21.3 |
| >80 | 320 + 1600 | | 4 | | 20 |

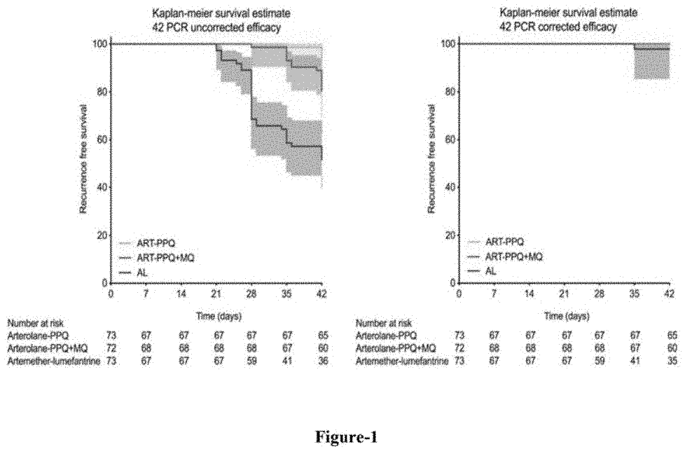

Further efficacy results are shown in Table 7 and FIG. 1, depicting that the present body weight-based dosing regimen endows a better alternative therapy in malaria control with a potential role in treatment of resistant malaria and prevention of further development of parasite resistance.

TABLE 7
43 day and 28 day APCR (PCR corrected and uncorrected)

| | | ART- | | AL vs | | ART-PPQ vs |
|---|---|---|---|---|---|---|
| | AL | PPQ + MQ | ART-PPQ | ART- | AL vs | ART- |
| | %, n/N | %, n/N | %, n/N | PPQ + MQ | ART-PPQ | PPQ + MQ |
| | (95% CI) | (95% CI) | (95% CI) | Risk difference (95% CI) (p-value) | | |
| PCR corrected | 98.6 | 100.0 | 100.0 | 1.4 | 1.4 | 0 |
| ACPR at day 42 | (72/73) | (72/72) | (73/73) | (−1.3 to 4.1) | (−1.3 to 4.1) | (NA) |
| (n/N, %) | (92.6 to 100.0) | (95.0 to 100.0) | (95.1 to 100.0) | 1.00 | 1.00 | NA |
| (intention to treat) | | | | | | |
| PCR uncorrected | 50.7 | 77.8 | 90.4 | 27.1 | 39.7 | 12.6 |
| ACPR at day 42 | (37/73) | (56/72) | (66/73) | (−12.1 to 42.1) | (26.4 to 53.0) | (0.9 to 24.3) |
| (n/N, %) | (38.7 to 62.6) | (66.4 to 86.7) | (81.2 to 96.1) | 0.001 | <0.0001 | 0.043 |
| (intention to treat) | | | | | | |
| PCR corrected | 97.2 | 100.0 | 100.0 | 2.8 | 2.8 | 0 |
| ACPR at day 42 | (35/36) | (60/60) | (65/65) | (−2.6 to 8.2) | (−2.6 to 8.2) | (NA) |
| (n/N, %) | (85.5 to 99.9) | (94.0 to 100.0) | (94.5 to 100.0) | 0.375 | 0.356 | NA |
| (per protocol) | | | | | | |
| PCR uncorrected | 49.2 | 79.4 | 93.9 | 30.2 | 44.7 | 14.5 |
| ACPR at day 42 | (32/65) | (54/68) | (62/66) | (14.7 to 45.7) | (31.2 to 58.2) | (3.3 to 25.7) |
| (n/N, %) | (36.6 to 61.9) | (67.9 to 88.3) | (85.2 to 98.3) | 0.0003 | <0.0001 | 0.0003 |
| (per protocol) | | | | | | |
| PCR corrected | 100.0 | 100.0 | 100.0 | 0 | 0 | 0 |
| ACPR at day 28 | (73/73) | (72/72) | (73/73) | (NA) | (NA) | (NA) |
| (n/N, %) | (95.1 to 100.0) | (95.0 to 100.0) | (95.1 to 100.0) | NA | NA | NA |
| (intention to treat) | | | | | | |
| PCR uncorrected | 67.1 | 95.8 | 95.9 | 28.7 | 28.8 | 0.1 |
| ACPR at day 28 | (49/73) | (69/72) | (70/73) | (17.0 to 40.4) | (17.1 to 40.5) | (−6.4 to 6.6) |
| (n/N, %) | (55.1 to 77.7) | (88.3 to 99.1) | (88.5 to 99.1) | <0.0001 | <0.0001 | 1.00 |
| (intention to treat) | | | | | | |
| PCR corrected | 100.0 | 100.0 | 100.0 | 0 | 0 | 0 |
| ACPR at day 28 | (44/44) | (67/67) | (67/67) | (NA) | (NA) | (NA) |
| (n/N, %) | (92.0 to 100.0) | (94.6 to 100.0) | (94.6 to 100.0) | NA | NA | NA |
| (per protocol) | | | | | | |
| PCR uncorrected | 64.6 | 98.5 | 100.0 | 33.9 | 35.4 | 1.5 |
| ACPR at day 28 | (42/65) | (67/68) | (66/66) | (21.9 to 45.9) | (23.8 to 47.0) | (−1.4 to 4.4) |
| (n/N, %) (per | (51.8 to 76.1) | (92.1 to 100.0) | (94.6 to 100.0) | <0.0001 | <0.0001 | 1.00 |
| protocol) | | | | | | |

P values represent two-sided Fisher’s exact tests.
AL: Artemether-Lumefantrine; ARTP-PPQ + MQ: Arterolane-Piperaquine plus Mefloquine; ART-PPQ: Arterolane-Piperaquine; CI: Confidence interval.

Observation from Table-7:

The 42-day PCR corrected efficacy was 100.0% ((73/73) 95% CI 95.1-100.0) after arterolane-piperaquine, 100.0% ((72/72) 95% CI 95.0-100.0) after arterolane-piperaquine-mefloquine and 98.6% ((72/73) 95% CI 92.6-99.9) after artemether-lumefantrine. Day 42 PCR corrected efficacy was not different between the three arms.

The presently disclosed dosage form is a novel fixed dose combination (FDC) of arterolane maleate and piperaquine phosphate. Arterolane (also known as OZ277), a trioxolane that is easy to manufacture using synthetic processes, is a rapidly acting blood schizonticide. The partner drug piperaquine, on the other hand, has a longer terminal half-life, offering an advantage of good ‘post treatment protection’ by killing any residual parasite.

Further, results from the Ring Stage Assay (RSA) conducted as per WHO's recommendation demonstrated that at 700 nM, arterolane was found to be extremely active against early ring stages from both artemisinin-resistant and artemisinin-sensitive parasites and demonstrated no cross-resistance to DHA, indicating that arterolane could be effective against artemisinin-resistant P. falciparum.

The efficacy of Synriam in the treatment of uncomplicated P. falciparum and P. vivax malaria has been established in multiple clinical studies in adults and children. Table 8 below summarizes key efficacy data from Phase II and III studies of Synriam. The efficacy data from a study conducted in Kenyan children with uncomplicated falciparum malaria, using the weight-based dosage regimen of arterolane and piperaquine (similar to the proposed higher dosage of Disclosed dosage form).

TABLE 8
Summary of Clinical Efficacy Data of Synriam and Disclosed
Formulation Dosing regimen (S⁺).

| | | Efficacy Results | | |
|---|---|---|---|---|
| Phase, Number of | | FCT | PCT | Other parasitological parameters |
| Patients | | Median | Median | |
| Population, Countries | Design, Treatment | (h) | (h) | |

Synriam in uncomplicated P. falciparam malaria

| | | | | |
|---|---|---|---|---|
| Phase II, N=240 | Randomized, open | 24 vs. 24 | 30 vs. 30 | PCR corrected ACPR (%) |
| Adults, children >12 - 65 | label, multi-centric | | | PP: 100 vs 98.7 (D28) |
| Y | AM+PQP (150/750 | | | ITT: 94.4 vs. 96.3 (D28) |
| India, Thailand | mg) vs. Coartem | | | Recrudescence: 0 vs 1 Subject |
| Phase II, N=141 | Open label, single | 10 | 24 | PCR corrected ACPR (%) |
| Children 6 months - 12 Y | arm, multi-centric. | | | PP: 100 (D 28) |
| India, Ivory Coast, | AM+PQP | | | ITT: 87.9 (D 42) |
| Rwanda | (37.5/187.5 mg)* | | | No recrudescence |
| Phase III, N=1073 | Randomized, | 6 vs. 12 | 24 vs. 24 | PCR corrected ACPR (%) D 28 |
| Adults, children >12 - 65 | double blind, multi- | | | PP: 99.25 vs. 99.1 |
| Y | centric | | | ITT: 92.86 vs. 92.46 |
| INDIA, Thailand, Mali, | AM+PQP (150/750 | | | PCR corrected ACPR (%)D 42 |
| Bangladesh, Ivory Coast, | mg) vs. Coartem | | | PP: 98.61 vs. 98.36 |
| Senegal, DRC, Malawi, | | | | ITT: 90.48 vs. 91.34 |
| Mozambique | | | | Recrudescence: 9 vs 5 subjects |
| | | | | Gametocyte clearance D42: comparable |
| Phase III, N=859 | Randomized, open | 6 vs. 12 | 24 vs. 24 | PCR corrected ACPR (%)D 28 |
| Children 6 months to 12 Y | label, multi-centric | | | PP: 100 vs. 98.5 |
| India, Mali, Ivory Coast, | AM+PQP | | | ITT: 96.0 vs. 95.8 |
| Senegal, DRC, Malawi, | (37.5/187.5 mg)* | | | PCR corrected ACPR (%)D 42 |
| Mozambique | vs. Coartem | | | PP: 99.8 vs. 98.8 |
| | | | | ITT: 94.4 vs. 93.1 |
| | | | | Recrudescence: 1 vs. 7 subjects |
| | | | | Gametocyte clearance D 42: comparable |

Synrian in uncomplicated P.viv ax malaria

| | | | | |
|---|---|---|---|---|
| Phase III, N=317 | Randomized, open | 24 vs. 24 | 24 vs. 26 | Proportion of aparasitemic & afebrile |
| Adults, Children >12 - 65 | label, multi-centric | | | patients at 72 h (%) |
| Y | AM+PQP (150/750 | | | PP: 100 vs. 99.3 |
| India | mg) vs. | | | ITT: 96.9 vs. 98.7 |
| Phase III, N=164 | Chloroquine | | | |
| Children 6 months - 12 Y | Randomized, open | 12 vs. 18 | 21 vs. 24 | Proportion of aparasitemic & afebrile |
| India | label, multi-centric. | | | patients at 72 h (%) |
| | AM+PQP (37.5 | | | PP: 100 vs. 100 |
| | /187.5 mg)* vs. | | | ITT: 98.2 vs. 100 |
| | Chloroquine | | | |

St in uncomplicated P.falciparam malaria

| | | | | |
|---|---|---|---|---|
| N=217 | Rndomized, open | 36 vs. 36 | 11 vs. 13 | D42 PCR corrected efficacy(%): 100 vs |
| Children 2 - 12 Y | label, single center | | | 96; AM+PQP non-inferior to Coartem |
| Kenya | AM + PQP (~4 | | | D42 PCR uncorrected efficacy (%): 90 vs |
| | mg/kg + ~18 | | | 50. Longer post-treatment prophylactic |
| | mg/kg) vs. Coartem | | | effect by AM+ PQP compared to |
| | | | | Coartem |

The clinical data suggested promising AUC and Cmax values which further supports that the presently disclose body weight based dosing regimen may be better and should provide a longer post-treatment prophylactic effect.

### Result

The study results depict that the presently disclosed body weight based dosing regimen offers comparatively efficacious therapy over existing products for the treatment of uncomplicated malaria. Arterolane-piperaquine and arterolane-piperaquine+mefloquine are highly effective, safe and well tolerated treatments for uncomplicated falciparum malaria in African children. Deploying arterolane based triple combinations could delay the development of antimalarial drug resistance against arterolane and its partner drugs.

Therefore from the above results as shown in example, it is believed that the presently disclosed body weight based dosing regimen of Arterolane and Piperaquine optionally with any additional existing anti-malarial drugs/combinations will be effective and provides a significant exposure of anti-malarial drugs for effective therapy in all patient groups both against sensitive and resistant malaria.

What is claimed is:

1. A composition for treating malaria comprising Arterolane and Piperaquine, wherein the Arterolane and Piperaquine is present in an effective amount according to a body-weight dosing regimen of a patient, wherein the dose of Arterolane ranges from about 3 mg/kg/day to about 6.5 mg/kg/day and the dose of Piperaquine ranges from about 15 mg/kg to about 32 mg/kg/day.

2. The composition of claim 1, wherein the dosage is administered in a fixed dose pharmaceutical composition in accordance with a body weight of a patient.

3. The composition of claim 1, wherein the therapeutically effective amount of Arterolane ranges from about 30 mg to about 350 mg and the therapeutically effective amount of Piperaquine ranges from about 150 mg to about 2000 mg.

4. The composition of claim 1, wherein the therapeutically effective amount of Arterolane ranges from about 30 mg to about 50 mg and the therapeutically effective amount of Piperaquine from about 150 mg to about 250 mg in accordance with a body weight of a patient ranging from 5 kg to up to 11 kg.

5. The composition of claim 1, wherein the therapeutically effective amount of Arterolane ranges from about 60 mg to about 100 mg and the therapeutically effective amount of Piperaquine ranges from about 300 mg to about 500 mg in accordance with a body weight of a patient ranging from 11 kg to up to 25 kg.

6. The composition of claim 1, wherein the therapeutically effective amount of Arterolane ranges from about 120 mg to about 200 mg and the therapeutically effective amount of Piperaquine ranges from about 600 mg to about 1000 mg in accordance with a body weight of a patient ranging from 25 kg to up to 60 kg.

7. The composition of claim 1, wherein the therapeutically effective amount of Arterolane ranges from about 250 mg to about 350 mg and the therapeutically effective amount of Piperaquine ranges from about 1275 mg to about 2000 mg in accordance with a body weight of a patient ranging from 60 kg to above 80 kg.

8. The composition of claim 1, wherein the dosing regimen comprises administering to the patient Arterolane at about 30 mg to about 50 mg and Piperaquine at about 150 mg to about 250 mg once-a-day for three days.

9. The composition of claim 8, wherein the patient has a body weight of 5 kg to up to 11 kg.

10. The composition of claim 1, wherein the dosing regimen comprises administering to the patient Arterolane at about 60 mg to about 100 mg and Piperaquine at about 300 mg to about 500 mg once-a-day for three days.

11. The composition of claim 10, wherein the patient has a body weight of 11 kg to up to 25 kg.

12. The composition of claim 1, wherein the dosing regimen comprises administering to the patient Arterolane at about 120 mg to about 200 mg and Piperaquine at about 600 mg to about 1000 mg once-a-day for three days.

13. The composition of claim 12, wherein the patient has a body weight of 25 kg to up to 60 kg.

14. The composition of claim 1, wherein the dosing regimen comprises administering to the patient Arterolane at about 250 mg to about 350 mg and Piperaquine at about 1275 mg to about 2000 mg once-a-day for at least three days.

15. The composition of claim 14, wherein the patient has a body weight of 60 kg to above 80 kg.

16. The composition of claim 2, wherein the fixed dose pharmaceutical composition comprises about 32 mg of Arterolane and about 160 mg of Piperaquine, wherein the fixed dose is administered to the patient once-a-day for three days when the body weight of the patient ranges from 5 kg to up to 8 kg.

17. The composition of claim 2, wherein the fixed dose pharmaceutical composition comprises about 48 mg of Arterolane and about 240 mg of Piperaquine and is administered to the patient once-a-day for three days when the body weight of the patient ranges from 8 kg to up to 11 kg.

18. The composition of claim 2, wherein the fixed dose pharmaceutical composition comprises about 64 mg of Arterolane and about 320 mg of Piperaquine and is administered to the patient once-a-day for three days when the body weight of the patient ranges from 11 kg to up to 17 kg.

19. The composition of claim 2, wherein the fixed dose pharmaceutical composition comprises about 96 mg of Arterolane and about 480 mg of Piperaquine and is administered to the patient once-a-day for three days therapy when the body weight of the patient ranges from 17 kg to up to 25 kg.

20. The composition of claim 2, wherein the fixed dose pharmaceutical composition comprises about 128 mg of Arterolane and about 640 mg of Piperaquine and is administered to the patient once-a-day for three days when the body weight of the patient ranges from 25 kg to up to 36 kg.

21. The composition of claim 2, wherein the fixed dose pharmaceutical composition comprises about 192 mg of Arterolane and about 960 mg of Piperaquine and is administered to the patient once-a-day for three days when the body weight of the patient ranges from 36 kg to up to 60 kg.

22. The composition of claim 2, wherein the fixed dose pharmaceutical composition comprises about 256 mg of Arterolane and about 1280 mg of Piperaquine and is administered to the patient once-a-day for three days when the body weight of the patient ranges from 60 kg to up to 80 kg.

23. The composition of claim 2, wherein the fixed dose pharmaceutical composition comprises about 320 mg of Arterolane and about 2000 mg of Piperaquine and is administered to the patient once-a-day for three days when the body weight of the patient is 80 kg and above.

24. The composition of claim 1, wherein the dosage form is a solid oral dosage form, liquid oral dosage form or an intravenous/parenteral dosage form.

25. The composition of claim 1, wherein the dosage is administered in conjunction with at least one additional therapeutically effective anti-malarial compound.

26. The composition of claim 25, wherein the additional therapeutically effective anti-malarial compound is Mefloquine or Primaquine.

27. The composition of claim 25, wherein a therapeutically effective amount of the at least one additional therapeutically effective anti-malarial compound is administered simultaneously, concurrently or concomitantly.

28. A method of treating malaria in a patient comprising administering to the patient a therapeutically effective amount of Arterolane and Piperaquine in accordance with a body weight-based dosing regimen, wherein the dose of Arterolane ranges from about 3 mg/kg to about 6.5 mg/kg and the dose of Piperaquine ranges from about 15 mg/kg to about 32 mg/kg.

29. A pharmaceutical kit comprising (i) Arterolane, (ii) Piperaquine, and (iii) instructions for administering a therapeutically effective amount of Arterolane and Piperaquine in accordance with a body weight-based dosing regimen, wherein the dose of Arterolane ranges from about 3 mg/kg/day to about 6.5 mg/kg/day and the dose of Piperaquine from about 15 to about 32 mg/kg.
